(19) United States
(12) Patent Application Publication
(10) Pub. No.: US 2023/0330090 A1
(43) Pub. Date: Oct. 19, 2023

(54) Compositions Comprising Lithium Orotate And L-Leucine And Methods For Improving Cognitive Performance

(71) Applicant: Jessica Dietrich-Marsh, Birmingham, AL (US)

(72) Inventor: Jessica Dietrich-Marsh, Birmingham, AL (US)

(21) Appl. No.: 18/303,227

(22) Filed: Apr. 19, 2023

Related U.S. Application Data
(60) Provisional application No. 63/332,538, filed on Apr. 19, 2022.

(51) Int. Cl. A61K 31/513; A61K 31/198; A61K 9/48

(52) U.S. Cl. CPC A61K 31/513; A61K 31/198; A61K 9/4816; A61K 9/4858

(57) ABSTRACT

The disclosure relates to compositions comprising lithium orotate and L-leucine encapsulated in a hypromellose capsule and methods of improving cognitive performance.

### CROSS REFERENCE TO RELATED APPLICATIONS

This application claims the benefit of priority of U.S. Provisional Application No. 63/332,538, filed Apr. 19, 2022. The content of these earlier filed applications is hereby incorporated by reference herein in their entirety.

### BACKGROUND

Cognitive disorders are associated with persistent and residual symptoms that can significantly impact a patient's functioning and quality of life. Many therapeutic agents associated with enhancing cognition are associated with untoward effects that can limit their use. Thus, therapeutic agents that can improve cognitive performance with a low side effect profile are needed.

### SUMMARY

Disclosed herein are compositions consisting of lithium orotate and L-leucine encapsulated in a hypromellose capsule.

Disclosed herein are compositions comprising lithium orotate and L-leucine encapsulated in a hypromellose capsule.

Disclosed herein are compositions consisting of lithium orotate, L-leucine, and one or more fish oils encapsulated in a hypromellose capsule.

Disclosed herein are compositions comprising lithium orotate, L-leucine, and one or more fish oils encapsulated in a hypromellose capsule.

Disclosed herein are methods for improving cognitive performance in a subject in need thereof, the methods comprising administering to the subject an effective amount of a composition consisting of lithium orotate and L-leucine encapsulated in a hypromellose capsule.

Disclosed herein are methods for improving cognitive performance in a subject in need thereof, the methods comprising administering to the subject an effective amount of a composition consisting of lithium orotate, L-leucine, and one or more fish oils encapsulated in a hypromellose capsule.

Other features and advantages of the present compositions and methods are illustrated in the description below, the drawings, and the claims.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or ‘approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease, disorder or condition. The term “patient” includes human and veterinary subjects. In some aspects of the disclosed methods, the “patient” has been diagnosed with a need for treatment for preventing or treating or improving cognitive performance, such as, for example, prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be concussion, a traumatic brain injury or other cognitive injury, or depression.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

Compositions

Disclosed herein are compositions comprising lithium orotate and L-leucine encapsulated in a hypromellose capsule. Also disclosed herein are compositions consisting of lithium orotate and L-leucine encapsulated in a hypromellose capsule. In some aspects, the composition can be formulated as an oral dosage form. In some aspects, the composition can be formulated as single oral dosage form. In some aspects, the lithium orotate can be in the amount of about 10 mg. In some aspects, the lithium orotate can be in the amount of 10 mg. In some aspects, the L-leucine can be in the amount of about 250 mg. In some aspects, the composition does not comprise microcrystalline cellulose, branched-chain amino acids other than l-leucine (e.g., isoleucine and valine), ascorbyl palmitate or silica (e.g., amorphous silica (SiO₂)).

Disclosed herein compositions comprising lithium orotate, L-leucine, and one or more fish oils encapsulated in a hypromellose capsule. Also disclosed herein are compositions consisting of lithium orotate, L-leucine, and one or more fish oils encapsulated in a hypromellose capsule. In some aspects, the composition can be formulated as an oral dosage form. In some aspects, the composition can be formulated as single oral dosage form. In some aspects, the lithium orotate can be in the amount of about 10 mg. In some aspects, the lithium orotate can be in the amount of 10 mg. In some aspects, the L-leucine can be in the amount of about 250 mg. In some aspects, one or more fish oils can be anchovy docosahexaenoic acid (DHA), sardine DHA, docosapentaenoic acid (DPA), eicosapentaenoic acid (EPA) or a combination thereof. In some aspects, the amount of anchovy DHA and sardine DHA can be equal. In some aspects, the composition does not comprise microcrystalline cellulose, branched-chain amino acids other than l-leucine (e.g., isoleucine and valine), ascorbyl palmitate or silica (e.g., amorphous silica (SiO₂)). In some aspects, the fish oils can comprise a fish oil concentrate. In some aspects, the fish oils can be derived from a fish oil concentrate. In some aspects, the fish oil concentrate can comprise a fish oil concentrate comprising anchovy DHA and sardine DHA in equal amounts. In some aspects, the amount of the fish oil concentration can be about 1220 mg. In some aspects, the composition can comprise a fish oil concentrate (from anchovy and sardine equally) that comprises DHA 400 mg, DPA 30 mg, and/or EPA 500 mg. In some aspects, the anchovy DHA can be in the amount of 800 mg; the sardine DHA can be in the amount of 800 mg; the DPA can be in the amount of 60 mg; and the EPA can be in the amount of 1000 mg.

In some aspects, the compositions can further comprise rosemary or astaxanthin.

Disclosed herein are compositions comprising or consisting of lithium orotate, one or more fish oils, rosemary and/or astaxanthin encapsulated in a hypromellose capsule. In some aspects, the composition can be formulated as an oral dosage form. In some aspects, the composition can be formulated as single oral dosage form. In some aspects, the lithium orotate can be in the amount of about 10 mg. In some aspects, the lithium orotate can be in the amount of 10 mg. In some aspects, one or more fish oils can be anchovy docosahexaenoic acid (DHA), sardine DHA, docosapentaenoic acid (DPA), eicosapentaenoic acid (EPA) or a combination thereof. In some aspects, the amount of anchovy and sardine DHA can be equal. In some aspects, the composition does not comprise microcrystalline cellulose, branched-chain amino acids other than l-leucine (e.g., isoleucine and valine), ascorbyl palmitate or silica (e.g., amorphous silica (SiO₂)). In some aspects, the compositions do not comprise L-leucine.

In some aspects, the compositions disclosed herein can further comprise rosemary. In some aspects, the compositions disclosed herein can further comprise astaxanthin.

In some aspects, the lithium orotate can be present in an amount up to about 10 mg. In some aspects, the lithium orotate can be present in an amount of 1 mg to 150 mg or any amount in between.

In some aspects, the compositions disclosed herein can be formulated as a capsule for oral administration.

In some aspects, the compositions disclosed herein can be used in combination with other therapeutic drugs used to treats subjects suffering from cognitive impairment, a concussion or depression.

Methods of Treatment

Disclosed herein are methods for improving cognitive performance in a subject in need thereof. In some aspects, the methods can comprise administering to the subject an effective amount of a composition consisting of lithium orotate and L-leucine encapsulated in a hypromellose capsule. In some aspects, the composition can be formulated as an oral dosage form. In some aspects, the composition can be formulated as single oral dosage form. In some aspects, the lithium orotate can be in the amount of about 10 mg. In some aspects, the lithium orotate can be in the amount of 10 mg. In some aspects, the L-leucine can be in the amount of about 250 mg. In some aspects, one or more fish oils can be anchovy docosahexaenoic acid (DHA), sardine DHA, docosapentaenoic acid (DPA), eicosapentaenoic acid (EPA) or a combination thereof. In some aspects, the amount of anchovy and sardine DHA can be equal. In some aspects, the composition does not comprise microcrystalline cellulose, branched-chain amino acids other than l-leucine (e.g., isoleucine and valine), ascorbyl palmitate or silica (e.g., amorphous silica (SiO₂)).

In some aspects, the subject is a human. In some aspects, the subject has been diagnosed as having a cognitive impairment prior to the administering step. In some aspects, the subject has been diagnosed as having or have had a concussion. In some aspects, the subject has been diagnosed as having or suffering from depression.

The compositions and formulations described herein can be formulated to be in a unit dosage formulation. In some aspects, compositions described herein can be administered on an as-needed basis.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to depression, a concussion or a cognitive impairment or the need for cognitive improvement.

The compositions described herein can be administered to the subject (e.g., a human patient) in an amount sufficient to delay, reduce, prevent or reverse the onset or duration of one or more symptoms of a concussion, depression or cognitive impairment in a subject. Accordingly, in some aspects, the patient can be a human patient. In therapeutic applications, compositions and formulations described herein can be administered to a subject (e.g., a human patient) already expressing or diagnosed with a cognitive impairment, concussion or depression or is need of improving cognitive performance in an amount sufficient to at least partially improve one or more symptoms of or associated with cognitive impairment, concussion or depression, its complications, and consequences. An amount adequate to accomplish this is defined as a therapeutically effective amount. A therapeutically effective amount of a composition can be an amount that achieves a cure or reverses one or more symptoms of cognitive impairment, concussion or depression or improves one or more symptoms of cognitive impairment, concussion or depression, but that outcome is only one among several that can be achieved. As noted, a therapeutically effect amount includes amounts that provide a treatment in which the onset, progression or expression of one or more of the side effects associated with cognitive impairment, concussion or depression can be delayed, hindered, or prevented, or the one or more symptoms associated with cognitive impairment, concussion or depression can be reduced, ameliorated or reversed. One or more of the symptoms can be less severe. Recovery can be accelerated in an individual who has been treated. In some aspects, the one or more side effects associated with cognitive impairment, concussion or depression include but are not muscle weakness, loss of appetite, mild apathy, listlessness, limited attention, executive functions, memory and processing speed.

Amounts effective for this use can depend on the severity of the cognitive impairment, concussion or depression and the weight and general state and health of the subject, but generally range from about 10 mg of an equivalent amount of the lithium orotate per dose per subject.

The total effective amount of any of the compositions disclosed herein can be administered to a subject as a single dose, either as a bolus or by infusion over a relatively short period of time, or can be administered using a fractionated treatment protocol in which multiple doses are administered over a more prolonged period of time.

The therapeutically effective amount or dosage of the compositions used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of side effects of sialorrhea or the sialorrhea condition itself), the age and physical characteristics of the subject and other considerations known to those of ordinary skill in the art.

As disclosed herein, the compositions disclosed herein can be formulated for oral administration. In preparing the composition for oral dosage form, the oral solid preparation can be in the form of a capsule. In some aspects, the capsule can be a hypromellose capsule.

The compositions disclosed herein can be administered directly to a subject.

The compositions can be sterile and sterilized by conventional sterilization techniques or sterile filtered. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tubes.

Dosage

The routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

In some aspects, the administration of the compositions disclosed herein can be by oral administration. In some aspects, the composition can administered about 24 hours apart. In some aspects, the composition can be administered once, twice or three times in a 24 hour period. In some aspects, the composition can be administered between 10 mg/kg/day to 30 mg/kg/day.

### ARTICLES OF MANUFACTURE

The composition described herein can be packaged in a suitable container labeled, for example, for use as a therapy to reduce accumulation of saliva in the mouth or treat sialorrhea in a subject. Accordingly, packaged products (e.g., sterile containers containing the composition or stable gel formulations described herein and packaged for storage, shipment, or sale at concentrated or ready-to-use concentrations) and kits, including at least lithium as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, dosing cards for measuring the dose and/or applicators for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the composition therein should be administered (e.g., the frequency and route of administration), indications therefor, and other uses. The composition can be ready for administration (e.g., present in dose-appropriate units), and may include a pharmaceutically acceptable adjuvant, carrier or other diluent. Alternatively, the composition can be provided in a concentrated form with a diluent and instructions for dilution.

A variety of containers and packaging can be used for the oral administration of the compositions described herein, including but not limited to single-use, and the like. In some aspects, the containers and packages can have an easy-opening design.

1. A composition consisting of lithium orotate and L-leucine encapsulated in a hypromellose capsule.

2. The composition of claim 1, wherein the composition is formulated as an oral dosage form.

3. The composition of claim 1, wherein the composition is formulated as single oral dosage form.

4. The composition of claim 1, wherein the lithium orotate is in the amount of about 10 mg.

5. The composition of claim 1, wherein the L-leucine is in the amount of about 250 mg.

6. A composition consisting of lithium orotate, L-leucine, and one or more fish oils encapsulated in a hypromellose capsule.

7. The composition of claim 6, wherein the composition is formulated as an oral dosage form.

8. The composition of claim 6, wherein the composition is formulated as single oral dosage form.

9. The composition of claim 6, wherein the lithium orotate is in the amount of about 10 mg.

10. The composition of claim 6, wherein the L-leucine is in the amount of about 250 mg.

11. The composition of claim 6, wherein the one or more fish oils are anchovy docosahexaenoic acid (DHA), sardine DHA, docosapentaenoic acid (DPA), eicosapentaenoic acid (EPA) or a combination thereof.

12. The composition of claim 11, wherein the anchovy DHA is in the amount of 800 mg; the sardine DHA is in the amount of 800 mg; the DPA is in the amount of 60 mg; and the EPA is in the amount of 1000 mg.

13. The composition of claim 1, wherein the composition does not comprise microcrystalline cellulose, branched-chain amino acids other than l-leucine, ascorbyl palmitate or silica.

14. A method for improving cognitive performance in a subject in need thereof, the method comprising administering to the subject an effective amount of a composition consisting of lithium orotate and L-leucine encapsulated in a hypromellose capsule.

15. (canceled)

16. The method of claim 14, wherein the lithium orotate is in the amount of about 10 mg.

17. The method of claim 14, wherein the L-leucine is in the amount of about 250 mg.

18. (canceled)

19. (canceled)

20. The method of claim 14, wherein the composition does not comprise microcrystalline cellulose, branched-chain amino acids other than l-leucine, ascorbyl palmitate or silica.

21. (canceled)

22. (canceled)

23. (canceled)

24. (canceled)

25. The method of claim 14, wherein the composition is administered about 24 hours apart.

26. The method of claim 14, wherein the composition is administered between 10 mg/kg/day to 30 mg/kg/day.

27. The composition of claim 6, wherein the composition does not comprise microcrystalline cellulose, branched-chain amino acids other than l-leucine, ascorbyl palmitate or silica (e.g., amorphous silica (SiO₂))
